(19) United States
(12) Patent Application Publication
(10) Pub. No.: US 2026/0256670 A1
(43) Pub. Date: Sep. 3, 2026

(54) COMPOSITION CONTAINING PEPTIDES HAVING SKIN-LIGHTENING EFFECT

(71) Applicant: REMEDI CO., LTD., Incheon (KR)

(72) Inventors: Chanho PARK, Incheon (KR); Seongmin CHO, Incheon (KR); Ee Chan SONG, Incheon (KR); Yungyeong SHIN, Incheon (KR)

(73) Assignee: REMEDI CO., LTD., Incheon (KR)

(21) Appl. No.: 18/867,016

(22) PCT Filed: Apr. 7, 2023
(86) PCT No.: PCT/KR2023/004728

§ 371 Date: Nov. 19, 2024

(30) Foreign Application Priority Data
Aug. 31, 2022 (KR) 10-2022-0109704
Apr. 7, 2023 (KR) 10-2023-0045817

(51) Int. Cl. A61K 8/64; A61Q 19/02

(52) U.S. Cl. CPC A61K 8/64; A61Q 19/02

(57) ABSTRACT

The present disclosure relates to pharmaceutical agents, cosmetics, medical devices, and the like, containing novel skin-lightening functional peptides and the present inventors found that the RMNE1 peptide of SEQ ID NO.: 1 and a variant thereof exhibit low cytotoxicity as well as high cell permeability and skin penetrability and show an excellent skin-lightening effect.

### TECHNICAL FIELD

The present disclosure relates to a cosmetic composition or pharmaceutical composition containing a peptide having a skin-lightening activity. The peptide having human-derived skin-lightening activity may be applied to cosmetics, medicine, and the like.

### BACKGROUND ART

Melanin, which determines human skin color, is produced in skin cells called melanocytes and moves to epidermal cells called keratinocytes. Herein, melanin plays an important role by forming a hat-like structure around the nucleus, protecting genes from ultraviolet rays and protecting intracellular proteins by removing free radicals.

Melanogenesis refers to the biosynthesis of melanin pigment in melanocytes, which is regulated by the intracellular and extracellular environment. Melanin is a phenolic polymer that is a complex of black pigment and protein. When melanin is overproduced, blemishes and freckles may form on the skin, and skin cancer may also occur. Melanin is produced through enzymatic and non-enzymatic oxidation reactions of tyrosine in melanocytes present in the basal layer of the epidermis. Specifically, this melanin production is triggered by tyrosinase, which oxidizes tyrosine to dopaquinone, which is further converted to pheomelanin, a pale yellow/red compound, and eumelanin, which is light brown to black. In the skin, melanin affects pigmentation and protects the skin from ultraviolet rays and other causes of skin diseases, but abnormal accumulation of melanin may also lead to the formation of blemishes and freckles.

Skin hyperpigmentation diseases caused by melanin overproduction include genetic skin diseases such as vitiligo, Waardenburg syndrome, acquired skin diseases such as post-inflammatory white pityriasis, idiopathic guttate hypomelanosis, melasma, skin diseases caused by medications such as minocycline, bleomycin, busulfan, zidovudine, and skin diseases spread through infection.

Thus far, research on melanin production inhibitors has mainly focused on developing tyrosinase inhibitors, and arbutin, para-methoxyphenol, hydroquinone, and kojic acid have been used as whitening agents. However, these have the issue of causing side effects such as weak activity or causing cell degeneration or death, thereby damaging the original function of the cells.

### DETAILED DESCRIPTION OF INVENTION

### Technical Problems

An aspect of the present disclosure is directed to providing a cosmetic composition and/or pharmaceutical composition containing a new peptide that is safe for living bodies, has high cell permeability and skin penetrability, and has excellent lightening activity.

### Technical Solution

In order to address an issue associated with the related art, as a result of selecting and testing numerous human-derived candidate peptides, the present inventors have found that a peptide consisting of 17 amino acids of DEQERRRRRGRTRVRSE (SEQ ID NO.: 1) derived from a human neurogenin-1 protein (hereinafter, “RMNE1”) or variant peptides consisting of amino acids in which 4 to 17 consecutive amino acids or 1 to 13 consecutive or non-consecutive amino acids of an RMNE1 sequence are deleted, such as variants of the RMNE1 described in Table 2, exhibited low cytotoxicity as well as high cell permeability and artificial skin penetrability and showed an excellent lightening effect.

Unless otherwise defined, all terms used herein, including technical or scientific terms, have the same meaning as generally understood by those skilled in the art to which the present disclosure pertains.

### Effect of Invention

A peptide having skin-lightening activity of an embodiment of the present disclosure exhibited low cytotoxicity, cell permeability, skin penetrability, and skin-lightening activity.

Accordingly, the peptide having skin-lightening activity of an embodiment of the present disclosure and the composition containing the same can be used in pharmaceutical compositions and medical devices for the prevention or treatment of blemishes, freckles, senile pigmentation spots, or solar lentigines, in addition to whitening cosmetic compositions.

### BRIEF DESCRIPTION OF THE DRAWING

FIG. 1 is a graph showing the results of identifying the cell permeability of RMNE1 in cell line B16F10 using an RMNE1 peptide attached with fluorescent FITC.

FIG. 2 is a graph showing the results of measuring the penetration efficiency of Strat-M™ Membrane, an artificial skin, using the RMNE1 peptide attached with the fluorescent FITC.

FIG. 3 is a graph showing the relative concentration of melanin after treating the cell line B16F10 with melanocyte-stimulating hormone (α-MSH) to induce melanin formation, followed by treatment with RMNE1 peptide and Arubutin, a control group, at different concentrations, and culturing the same for 3 days.

FIG. 4 shows the results of evaluating the lightening effect of skin-lightening functional peptides of an embodiment of the present disclosure.

FIG. 5 shows the results of evaluating the efficacy of the skin-lightening functional peptide using artificial skin.

FIG. 6 shows the results of evaluating the synergy between the skin-lightening functional peptide of an embodiment of the present disclosure and vitamin C or niacinamide using artificial skin.

FIG. 7 shows the results of evaluating the synergy between the skin-lightening functional peptide of an embodiment of the present disclosure and vitamin C and niacinamide using artificial skin.

FIG. 8 shows the results of identifying the lightening effect by conjugating palmitoyl group to the skin-lightening functional peptides of RMNE1 and RMNE1-2 of an embodiment of the present disclosure.

### BEST MODE FOR CARRYING OUT THE INVENTION

An embodiment of the present disclosure relates to a cosmetic composition for skin-lightening, wherein the composition includes one or more skin-lightening functional peptides selected from:
a) an RMNE1 peptide consisting of an amino acid sequence of SEQ ID NO.: 1;
b) an RMNE1 peptide deletion variant having one to thirteen consecutive or non-consecutive amino acids deleted from the amino acid sequence of SEQ ID NO.: 1; and
c) an RMNE1 peptide deletion variant consisting of four to sixteen consecutive amino acids from the amino acid sequence of SEQ ID NO.: 1.

In b) above, the RMNE1 peptide variant preferably consists of 4 or more, or 5 or more, or 6 or more, or 7 or more, or 8 or more, or 9 or more, or 10 or more, or 11 or more, or 12 or more, or 13 or more, or 14 or more, or 15 or more, or 16 or more consecutive amino acids of SEQ ID NO.: 1.

In addition, an embodiment of the present disclosure relates to a cosmetic composition for skin-lightening, wherein the RMNE1 peptide deletion variant is not limited thereto, but is, for example, a peptide consisting of an amino acid sequence selected from SEQ ID NOs: 2 to 14. Data for SEQ ID NOs.: 10 to 14 is not shown in this specification, but the sequence of SEQ ID NO. 10, that is, RMNE1-9, is DEQERR, which may be generated by a combination of SEQ ID NO. 5 (DEQE) and SEQ ID NO. 9 (QERR); accordingly, the whitening function of SEQ ID NO.: 10 may be inferred by those skilled in the art. The sequence of SEQ ID NO.: 11, that is, RMNE1-10, is GRTRVR, which may be generated by a combination of SEQ ID NO.: 7 (GRTR) and SEQ ID NO.: 8 (TRVR); accordingly, the whitening function of SEQ ID NO.: 11 may be inferred by those skilled in the art. The sequence of SEQ ID NO.: 12, that is, RMNE1-11, is GRTRVRSE, which may be generated by a combination of SEQ ID NO.: 7 (GRTR), SEQ ID NO.: 8 (TRVR), and SEQ ID NO.: 6 (VRSE), or a combination of SEQ ID NO.: 7 (GRTR) and SEQ ID NO.: 6 (VRSE); accordingly, the whitening function of SEQ ID NO.: 12 may be inferred by those skilled in the art. In addition, the sequence of SEQ ID NO.: 13, that is, RMNE1-12, is ERRRRRGRTRVRSE, which may be generated by a combination of SEQ ID NO.: 4 (ERRRRRGRTRV) and SEQ ID NO.: 2 (RRRRRGRTRVRSE); accordingly, the whitening function of SEQ ID NO.: 13 may be inferred by those skilled in the art. In addition, the sequence of SEQ ID NO.: 14, that is, RMNE1-13, is QERRRRRGRTRVRSE, which may be generated by a combination of SEQ ID NO.: 9 (QERR) and SEQ ID NO.: 2 (RRRRRGRTRVRSE); accordingly, the whitening function of SEQ ID NO.: 14 may be inferred by those skilled in the art.

In addition, an embodiment of the present disclosure relates to a cosmetic composition for skin-lightening, wherein the skin-lightening functional peptide is a combination of two or more same or different skin-lightening functional peptides.

In addition, an embodiment of the present disclosure relates to a cosmetic composition for skin-lightening, wherein the skin-lightening functional peptide is characterized in that one or more of a functional molecule, a functional group, a biocompatible polymer, and a fatty acid are bound to at least one of an N-terminus and a C-terminus of the peptide.

In addition, the peptide according to an embodiment of the present disclosure may be modified with functional groups such as phosphorylation, sulfation, acrylation, glycosylation, methylation, and farnesylation.

In addition, the peptide or fragment thereof of an embodiment of the present disclosure may form a conjugate conjugated with a biocompatible polymer or fatty acid. The biocompatible polymers may be one or more selected from the group consisting of starch, dextran, chitosan, glycol chitosan, pullulan, chondroitin sulfate, hyaluronic acid, pectin, polylactic acid (PLA), polyglycolide (PGA), polycaprolactone (PCL), poly(caprolactone-lactide) random copolymer (PCLA), and the like, but is not limited thereto. In addition, the fatty acid may be one or more selected from the group consisting of hexanoic acid, caprylic acid, capric acid, lauric acid, palmitic acid, stearic acid, and cholesterol, but is not limited thereto.

In addition, in an embodiment of the present disclosure, the functional molecule is a therapeutic peptide or therapeutic protein, and more specifically, a molecule exhibiting antioxidant, anti-inflammatory or wound healing functions.

The skin-lightening functional peptide may be bound and/or the skin-lightening functional peptide and the functional molecule may be fused without a linker or in the presence of a linker. In addition, the linker is not particularly limited as long as the skin-lightening activity of the skin-lightening functional peptide and the original activity of the functional molecule are maintained, but preferably includes an amino acid such as glycine, alanine, leucine, isoleucine, proline, serine, threonine, asparagine, aspartic acid, cysteine, glutamine, glutamic acid, lysine, arginine acid, and the like, to link the cargo molecule transduction domain and the cargo molecule, more preferably amino acids selected from valine, leucine, aspartic acid, glycine, alanine, proline, and the like, to link the same using several linked linkers and most preferably 1 to 10 amino acids selected from glycine, valine, leucine, aspartic acid, and the like, in consideration of the ease of genetic manipulation, and preferably, 1 to 5 amino acids to be linked and used as linkers. In addition to the above amino acid linkers and peptide linkers, chemical linkers may also be used as long as the activities of the skin-lightening functional peptide and the functional molecule are maintained.

In addition, an embodiment of the present disclosure relates to a pharmaceutical composition for preventing or treating a melanin hyperpigmentation disease, wherein the melanin hyperpigmentation disease includes blemishes, freckles, senile pigmentation spots, or solar lentigines, wherein the composition includes one or more skin-lightening functional peptides selected from:
a) an RMNE1 peptide consisting of an amino acid sequence of SEQ ID NO.: 1;
b) an RMNE1 peptide deletion variant having one to thirteen consecutive or non-consecutive amino acids deleted from the amino acid sequence of SEQ ID NO.: 1; and
c) an RMNE1 peptide deletion variant consisting of four to sixteen consecutive amino acids from the amino acid sequence of SEQ ID NO.: 1.

In addition, an embodiment of the present disclosure relates to a pharmaceutical composition for preventing or treating a melanin hyperpigmentation disease, wherein the RMNE1 peptide deletion variant is any one selected from SEQ ID NOs: 2 to 14. Data for SEQ ID NOs.: 10 to 14 is not shown in this specification, but the sequence of SEQ ID NO. 10, that is, RMNE1-9, is DEQERR, which may be generated by a combination of SEQ ID NO. 5 (DEQE) and SEQ ID NO. 9 (QERR); accordingly, the whitening function of SEQ ID NO.: 10 may be inferred by those skilled in the art. The sequence of SEQ ID NO.: 11, that is, RMNE1-10, is GRTRVR, which may be generated by a combination of SEQ ID NO.: 7 (GRTR) and SEQ ID NO.: 8 (TRVR); accordingly, the whitening function of SEQ ID NO.: 11 may be inferred by those skilled in the art. The sequence of SEQ ID NO.: 12, that is, RMNE1-11, is GRTRVRSE, which may be generated by a combination of SEQ ID NO.: 7 (GRTR), SEQ ID NO.: 8 (TRVR), and SEQ ID NO.: 6 (VRSE), or a combination of SEQ ID NO.: 7 (GRTR) and SEQ ID NO.: 6 (VRSE); accordingly, the whitening function of SEQ ID NO.: 12 may be inferred by those skilled in the art. In addition, the sequence of SEQ ID NO.: 13, that is, RMNE1-12, is ERRRRRGRTRVRSE, which may be generated by a combination of SEQ ID NO.: 4 (ERRRRRGRTRV) and SEQ ID NO.: 2 (RRRRRGRTRVRSE); accordingly, the whitening function of SEQ ID NO.: 13 may be inferred by those skilled in the art. In addition, the sequence of SEQ ID NO.: 14, that is, RMNE1-13, is QERRRRRGRTRVRSE, which may be generated by a combination of SEQ ID NO.: 9 (QERR) and SEQ ID NO.: 2 (RRRRRGRTRVRSE); accordingly, the whitening function of SEQ ID NO.: 14 may be inferred by those skilled in the art.

In addition, an embodiment of the present disclosure relates to a pharmaceutical composition for preventing or treating a melanin hyperpigmentation disease, wherein the skin-lightening functional peptide is a combination of two or more same or different skin-lightening functional peptides.

In addition, an embodiment of the present disclosure relates to a pharmaceutical composition for preventing or treating a melanin hyperpigmentation disease, wherein the skin-lightening functional peptide is characterized in that one or more of a functional molecule, a functional group, a biocompatible polymer, and a fatty acid are bound to at least one of an N-terminus and a C-terminus.

In addition, an embodiment of the present disclosure relates to a pharmaceutical composition for preventing or treating a melanin hyperpigmentation disease, wherein the functional molecule is a therapeutic peptide or therapeutic protein.

In addition, an embodiment of the present disclosure relates to a pharmaceutical composition for preventing or treating a melanin hyperpigmentation disease, wherein the functional molecule is a molecule exhibiting antioxidant, anti-inflammatory or wound healing functions.

In addition, an embodiment of the present disclosure relates to a medical device selected from among wound dressings, fillers, or composite fillers including one or more skin-lightening functional peptides selected from:
a) an RMNE1 peptide consisting of an amino acid sequence of SEQ ID NO.: 1;
b) an RMNE1 peptide deletion variant having one to thirteen consecutive or non-consecutive amino acids deleted from the amino acid sequence of SEQ ID NO.: 1; and
c) an RMNE1 peptide deletion variant consisting of four to sixteen consecutive amino acids from the amino acid sequence of SEQ ID NO.: 1.

In the pharmaceutical composition and the medical device, the skin-lightening functional peptide may be used in a state in which two or more types of identical or non-identical peptides are combined (linked), and the skin-lightening functional peptide and the functional molecule may be used without a linker or in a state of being linked through the linker described above.

In addition, an embodiment of the present disclosure is characterized by excellent skin penetrability of the skin-lightening functional peptide.

In addition, in an embodiment of the present disclosure, the skin-lightening functional peptide is combined with two or more identical or different sequences, preferably 2 to 25, more preferably 2 to 20. For example, the RMNE1 peptide may be used alone or linked in the form of a dimer or trimer. In addition, for example, the RMNE1 peptide may be used as a dimer, trimer, and the like, linked with or without a linker between monomers.

In addition, an embodiment of the present disclosure is characterized in that the functional molecule is a therapeutic peptide including a hormone, growth factor, neurotransmitter, and ion channel ligand, or a therapeutic protein such as various enzymes including superoxide dismutase, albumin, and antibody.

In addition, an embodiment of the present disclosure is characterized in that the functional molecule is a molecule that exhibits an antioxidant function, such as peroxiredoxin, an anti-inflammatory function, such as an interleukin, or a wound healing function.

In addition, the cosmetics of an embodiment of the present disclosure may include color cosmetics such as foundation, lipstick, and eye shadow in addition to basic cosmetics such as lotion, cream, essence, oil-in-water or water-in-oil emulsion, and ointment.

As used herein, the term “conservative substitution” refers to modification of a cargo molecule transduction domain including substituting one or more amino acids by amino acids having similar biological or biochemical properties that do not cause loss of the biological or biochemical functions of the cargo molecule transduction domain.

As used herein, the term “conservative amino acid substitution” refers to a substitution to replace an amino acid residue by an amino acid residue having a similar side chain. Classes of the amino acid residue having a similar side chain are defined and well-known in the art. Such classes include amino acids with basic side chains (for example, lysine, arginine, histidine), amino acids with acidic side chains (for example, aspartic acid, glutamic acid), amino acids with uncharged polar side chains (for example, glycine, asparagine, glutamine, serine, threonine, tyrosine, cysteine), amino acids having non-polar side chains (for example, alanine, valine, leucine, isoleucine, proline, phenylalanine, methionine, tryptophan), amino acids having beta-branched side chains (for example, threonine, valine, isoleucine) and amino acids having aromatic side chains (for example, tyrosine, phenylalanine, tryptophan, and histidine).

The skin-lightening functional peptide according to an embodiment of the present disclosure is interpreted to include variants in which one or more amino acids in the RMNE1 peptide are mutated due to substitution, deletion, and/or addition.

In addition, the skin-lightening functional peptide or its variant according to an embodiment of the present disclosure has substantially the same function and/or effect as the skin-lightening functional peptide RMNE1 according to an embodiment of the present disclosure, and is interpreted to include peptide variants or fragments thereof having an amino acid sequence identity of 80% or 85% or more, preferably 90% or more, more preferably 95% or more, 96% or more, 97% or more, 98% or more, or 99% or more. In addition, the skin-lightening functional peptide RMNE1 and deletion variants thereof of an embodiment of the present disclosure as described above and variants having a sequence identity of 80% or more, or 85% or more, preferably 90% or more, more preferably 95% or more, more preferably 97% or more, 98% or more, or 99% or more thereto are expected to still possess skin-lightening activity despite conservative amino acid substitutions, amino acid deletions, or amino acid additions.

In addition, an embodiment of the present disclosure is characterized in that the functional molecule is selected from nucleic acids such as proteins, peptides, oligonucleotides, and polynucleotides, carbohydrates, lipids, and mixtures of one or more thereof.

In addition, an embodiment of the present disclosure is characterized in that the chemical bond between the functional molecule and the skin-lightening functional peptide is a covalent bond or a non-covalent bond, and is preferably a covalent bond. The non-covalent bonds may include ionic bonds, bonds due to electrostatic attraction, or bonds due to hydrophobic interactions. In addition, the material that may bind to the anti-inflammatory peptide through ionic bonds or electrostatic attraction may be a charged functional molecule such as DNA or RNA.

The skin-lightening functional peptide of an embodiment of the present disclosure is not limited to SEQ ID NOs: 1 to 14, but representative peptides are shown in Table 1 for convenience of experiment.

In addition, the pharmaceutical composition containing the skin-lightening functional peptide or the fusion compound of the skin-lightening functional peptide and the functional molecule of an embodiment of the present disclosure as an active ingredient may be mixed with a carrier commonly accepted in the pharmaceutical field and formulated in various forms such as an external skin preparation, oral administration, spray, patch, or injection by conventional methods. Examples of oral compositions include tablets and gelatin capsules, and may contain diluents (for example: lactose, dextrose, sucrose, mannitol, sorbitol, cellulose and/or glycine) and glydents (for example: silica, talc, and stearic acid and its magnesium or calcium salts, and/or polyethylene glycol) in addition to the active ingredients. In addition, the tablets may preferably contain binding agents such as magnesium aluminum silicate, starch paste, gelatin, methyl cellulose, sodium carboxymethyl cellulose and/or polyvinyl pyrrolidine, and depending on the situation, may contain disintegration agents such as starch, agar, alginic acid or its sodium salt or similar mixture and/or absorbent, coloring, flavor and sweetener. Compositions for injection are preferably isotonic aqueous solutions or suspensions, and the compositions mentioned are sterile and/or contain auxiliaries (for example, preservatives, stabilizers, wetting or emulsifying agents, solution accelerators, salts and/or buffers for adjusting osmotic pressure). In addition, these compositions may contain other therapeutically useful substances.

The pharmaceutical preparation prepared as such may be administered orally or parenterally, that is, via intravenous, subcutaneous, intraperitoneal, or topical administration, according to the intended purposes. The daily dosage of 0.0001-100 mg/kg may be administered once a day or a few times a day. The dosage level for a specific patient may vary depending on the patient's weight, age, gender, health condition, administration time, administration method, excretion rate, and severity of disease.

In addition, the pharmaceutical composition for preventing or treating a disease containing the skin-lightening functional peptide or the fusion compound of the skin-lightening functional peptide and the functional molecule as an active ingredient is an external skin preparation. The coating agent containing the composition to which the skin-lightening functional peptide and/or functional molecule of an embodiment of the present disclosure is added as an active ingredient may be easily prepared in any form according to a conventional preparation method. For example, in preparing a cream-type coating agent, the anti-inflammatory composition of an embodiment of the present disclosure is added to a general oil-in-water (O/W) or water-in-oil (W/O) cream base, to which perfumes, chelating agents, pigments, antioxidants, preservatives and the like may be used as needed, while synthetic or natural materials, such as proteins, minerals and vitamins, may be used in combination for the purpose of improving the physical properties thereof.

Other terms used in the specification, claims, and drawings, unless specifically specified, are used in the sense generally used by those skilled in the art in the technical field to which the present disclosure pertains.

TABLE 1

| Name | Amino acid sequence | SEQ ID NO. |
|---|---|---|
| RMNE1 | DEQERRRRRGRTRVRSE | 1 |
| RMNE1-1 | RRRRRGRTRVRSE | 2 |
| RMNE1-2 | DEQERRRRRGRTR | 3 |
| RMNE1-3 | ERRRRRGRTRV | 4 |
| RMNE1-4 | DEQE | 5 |
| RMNE1-5 | VRSE | 6 |
| RMNE1-6 | GRTR | 7 |
| RMNE1-7 | TRVR | 8 |
| RMNE1-8 | QERR | 9 |
| RMNE1-9 | DEQERR | 10 |
| RMNE1-10 | GRTRVR | 11 |
| RMNE1-11 | GRTRVRSE | 12 |
| RMNE1-12 | ERRRRRGRTRVRSE | 13 |
| RMNE1-13 | QERRRRRGRTRVRSE | 14 |

### MODE FOR CARRYING OUT THE INVENTION

Hereinafter, the configuration of the present disclosure will be described in more detail through specific examples. However, it is obvious to those skilled in the technical field to which the present disclosure pertains that the scope of the present disclosure is not limited to the description of the examples.

### 1. Cell Culture

HaCaT cells and B16F10 cells were cultured in a cell culture medium supplemented with 10% of fetal bovine serum (FBS) and antibiotic solution (100 units/ml penicillin, 100 μg/ml of streptomycin) in DMEM medium. The cells were cultured under conditions maintained at 37° C., 95% humidity, and 5% CO₂, and when the cells were 70-80% adhered to the culture dish, they were treated with trypsin-EDTA and subcultured. HaCaT cells were distributed and cultured from Professor Tae-Yoon Kim of the College of Medicine, The Catholic University of Korea, and B16F10 cells were distributed and cultured from the Korean Cell Line Bank.

### 2. Synthesis of Peptide RMNE1

The skin-lightening peptides of an embodiment of the present disclosure, such as RMNE1, were synthesized using a standard Fmoc-SPPS (solid phase peptide synthesis) protocol using 2-chlorotrityl chloride resin and amino acids. The peptide was then cleaved from the resin and side chain protecting groups were removed from the amino acid using TFA acid, water and triisopropylsilane. Ether was added for precipitation, and the peptide was purified using reverse-phase HPLC using a C18 column. Elution was performed with a water-acetonitrile linear gradient (0-75% (v/v) of acetonitrile) containing 0.1% (v/v) of TFA. To remove TFA, the peptide was dissolved in water and 100 mM of HCl was added to prepare HCl with a concentration of 8 mM. The solution was incubated at room temperature for 5 minutes, frozen in liquid nitrogen, lyophilized overnight to remove all liquid, and the lyophilized powder was redissolved in HCl solution, frozen again, and then lyophilized overnight. Freeze-drying was repeated for re-dissolution three times. After the final freeze-drying stage, the peptide was re-dissolved in water and the molecular weight of the purified peptide was checked using LC/MS.

### 3. Cytotoxicity Evaluation

To evaluate the toxicity of RMNE1 in HaCaT skin cells, 3×10⁴ of HaCaT cells were attached to a 96-well plate 12 hours ago. Thereafter, the cells were washed using serum-free DMEM medium and then treated with RMNE1 peptide at different concentrations in the serum-free medium. After 24 hours, the cells were washed a total of three times with serum-free medium, and then cell activity was measured using the CCK8 assay.

### 4. Cell Permeability Evaluation

To evaluate the cell permeability of RMNE1 in B16F10 cells, 1×10⁵ of B16F10 cells were attached to a 24-well plate 12 hours ago. Thereafter, the cells were washed using serum-free DMEM medium and then treated with 2.5 uM of peptide in the serum-free medium for 2 hours. After treatment, the cells were washed a total of three times with serum-free medium, and then the cells were dropped from the plate by treatment with 150 ul of trypsin, and then neutralized by adding 850 ul of serum-containing medium. Thereafter, after centrifugation at 500 g for 10 minutes, the medium was removed, and then 500 ul of FACS buffer (containing 1% of BSA and 0.1% of sodium azide) was added and FACS analysis was performed.

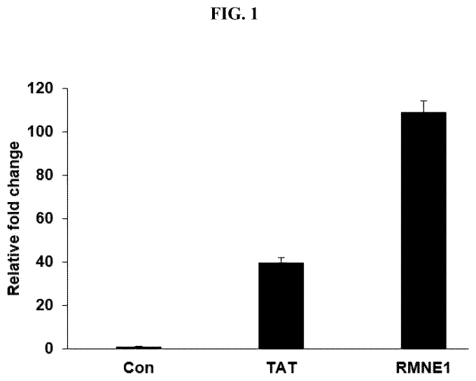

The cell permeability of RMNE1 into the cell line B16F10 was checked using a synthetic peptide attached with fluorescent FITC. FITC-attached peptides, RMNE1-FITC peptide, and TAT-FITC peptide were used at the same concentration of 2.5 uM for 2 hours. As a result, the RMNE1-FITC synthetic peptide showed a significantly higher cell penetration level compared to the control group, TAT-FITC peptide (FIG. 1).

### 5. Lightening Effect Evaluation

To evaluate the lightening effect of RMNE1 using B16F10 cells, the cells were attached to a 24-well plate so as to be filled to about 90%. Thereafter, the cells were washed with serum-free DMEM medium, and then 2% of FBS DMEM medium was prepared and 50 nM of melanocyte-stimulating hormone (α-MSH) was added. Then, RMNE1 was diluted to an appropriate concentration in the culture medium, and then the diluted culture medium was treated with the B16F10 cells and cultured for an additional 3 days.

Then, the culture medium was removed, the B16F10 cells were washed once with phosphate-buffered saline (PBS) solution, and then a solution containing 10% of DMSO in IN NaOH was added and reacted at 60° C. for 1 hour to lyse the cells. Thereafter, a portion of the cell lysate was transferred to a 96-well plate, and the absorbance was measured at a wavelength of 490 nm using a micro-plate reader (SpectraMax ABS Plus).

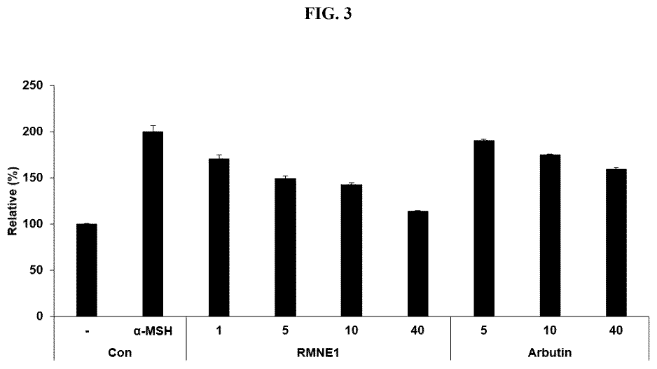

To check the lightening effect of the peptide, the B16F10 cell line was treated with melanocyte-stimulating hormone (α-MSH) to induce melanin formation, and then RMNE1 peptide and Arubutin, a control group, were treated at different concentrations and cultured for 3 days. To check the inhibition of melanin formation, the culture medium was removed, the cells were washed with the PBS solution, and melanin in the cells was dissolved by reacting with IN NaOH containing 10% of DMSO at 60° C. for 1 hour. Thereafter, as a result of reading using a microplate reader, melanin increased about two-fold in the test group treated with α-MSH compared to the untreated control group. The melanin production inhibition efficacy of RMNE1 was identified at each concentration, and a higher melanin inhibition efficacy was identified compared to the control group, Arbutin (FIG. 3).

### 6. Evaluation of Artificial Skin Penetrability of RMNE1

A Franz method was used to evaluate the skin penetrability of RMNE1. A 300 vm thick Strat-M™ Membrane was placed on the receptor chamber of the Franz diffusion cell device with the stratum corneum facing upward, and then the donor chamber was placed on the stratum corneum and fixed with a clamp. After filling a lower portion of the Franz diffusion cells with 8 ml of PBS, a receptor fluid, a magnetic bar was added and the receptor fluid was mixed homogeneously while maintaining 600 rpm. During the experiment, the skin and aqueous solution temperature was kept constant at 32±1° C. and humidity was 30-70%, and the experiment was conducted after stabilization for 30 minutes.

To evaluate skin penetrability, each test substance was labeled with FITC, and 1 mg/ml of the test substance was diluted in PBS at a ratio of 1:10, and then 1 ml of the diluted solution was applied to the donor chamber. To prevent volatilization of test substances through the donor chamber and sampling port, the entrance was sealed using parafilm. After 24 hours, 1 ml of receptor fluid was collected and diluted in 7 ml of PBS to analyze the test substances permeated in the receptor fluid. Fluorescence values were measured using a diluted fluorescence microplate reader.

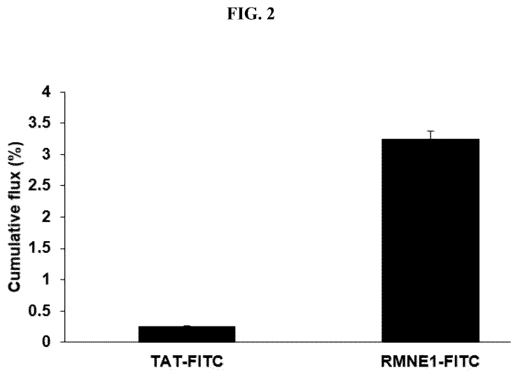

An experiment was conducted using a Franz diffusion cell device to measure the penetration efficiency of Strat-M™ Membrane, an artificial skin, using a synthetic peptide attached with fluorescent FITC. After placing the stratum corneum facing upward, the skin penetrability was observed for 24 hours using RMNE1-FITC peptide and TAT-FITC peptide, and the amount of permeated peptide was analyzed. As a result, the RMNE1-FITC peptide showed a significantly higher skin penetration level than the control group, TAT-FITC peptide, in both cell lines (FIG. 2).

### 7. Evaluation of Lightening Effect of RMNE1 Variants

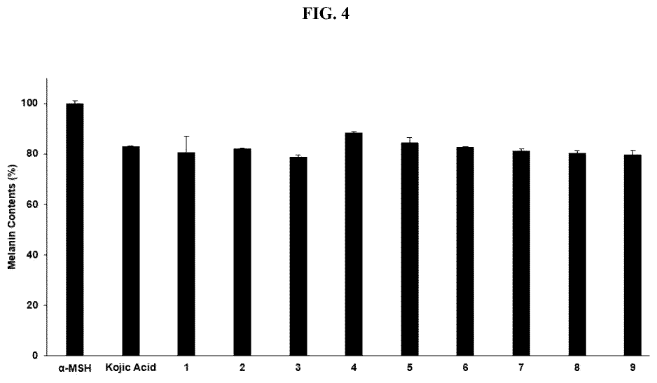

To check the lightening effect of the peptide, melanocyte-stimulating hormone (α-MSH) was treated with the B16F10 cell line to induce melanin formation, and then RMNE1 peptide, RMNE1 deletion variant peptide, and Kojic Acid as a control group were treated at different concentrations, and then were cultured for 3 days. To check inhibition of melanin formation, the culture medium was removed, washed with PBS solution, and melanin in the cells was dissolved by reacting with IN NaOH containing 10% of DMSO at 60° C. for 1 hour. Thereafter, the results were read using a microplate reader, and lightening effect was checked in RMNE1 and RMNE1 variants (FIG. 4).

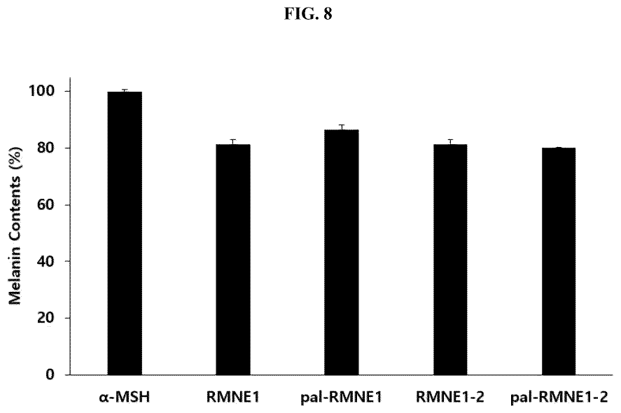

In addition, the lighting effect was checked depending on whether palmitoyl group conjugation (palmitoylation) was performed to the RMNE1 and RMNE1-2 peptides of an embodiment of the present disclosure. The peptides were treated at a concentration of 10 uM. Both RMNE1 (pal-RMNE1) and RMNE1-2 (pal-RMNE1-2) with palmitoyl groups conjugated were identified to maintain the same lightening effect compared to RMNE1-1 and RMNE1-2 peptides without palmitoyl groups conjugated (FIG. 8).

### 8. Evaluation of Efficacy of Skin-Lightening Functional Peptides Using Artificial Skin

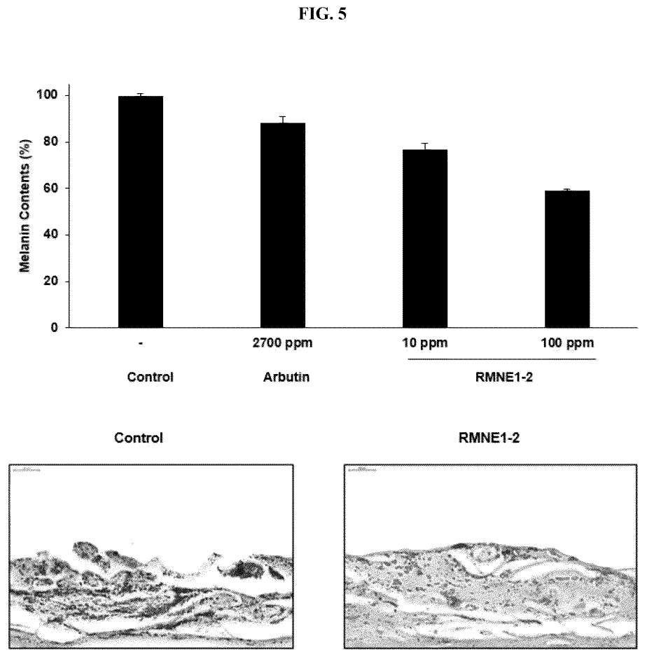

Using melanin-induced artificial skin (Neoderm-ME), an untreated control group, 2,700 ppm of arbutin, 10 ppm of RMNE1-2 peptide, and 100 ppm of RMNE1-2 peptide, was cultured by adding 1 ml of Neoderm-ME culture media containing 10% of a serum medium into each well and placing the same on a Neoderm-ME transwell. The peptides were treated twice on days 1 and 3, and on day 6, 50% of the artificial skin was partially dissolved in NaOH to measure the total melanin content of the artificial tissue. 50% of the artificial skin was made into a paraffin block, stained with fontana-masson, and photographed with an imaging microscope. The results are as shown in FIG. 5.

9. Evaluation of Synergy Effect Between Skin-Lightening Functional Peptides and Vitamin C and/or Niacinamide Using Artificial Skin

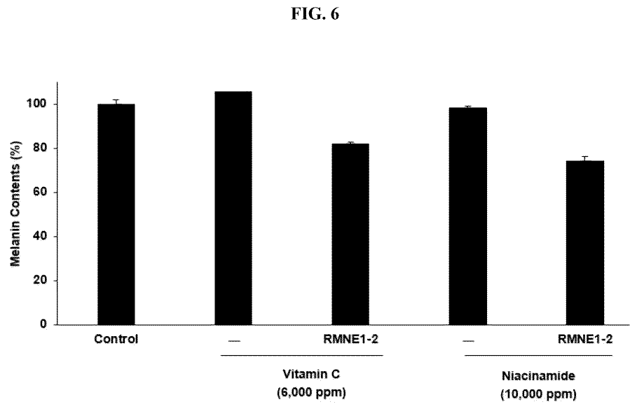

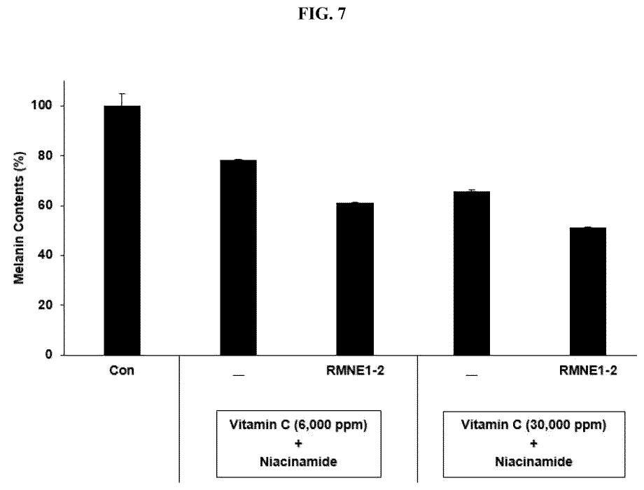

The synergy effect between the skin-lightening functional peptide RMNE1-2 of an embodiment of the present disclosure and vitamin C or niacinamide was evaluated using melanin-induced artificial skin (Neoderm-ME). After treatment with 6,000 ppm of vitamin C and 10,000 ppm of niacinamide, 2 ppm of RMNE1-2 was treated on days 1 and 3, respectively. On day 6, the tissue was dissolved in NaOH to measure the total melanin content of the artificial skin (FIG. 6). An evaluation experiment using artificial skin was conducted to evaluate the synergistic effect of skin-lightening functional peptide RMNE1-2 with vitamin C and niacinamide. The total melanin content of the artificial skin was measured on the 6th day after treatment with 1 ppm of RMNE1-2, 10,000 ppm of niacinamide, and 6,000 and 30,000 ppm of vitamin C for 1 and 3 days (FIG. 7).

### INDUSTRIAL APPLICABILITY

The skin-lightening functional peptide of an embodiment of the present disclosure is a part of human-derived protein and has low cytotoxicity, making it safe for the human body, and has a similar or better lightening effect compared to kojic acid, a representative whitening agent. Therefore, the skin-lightening functional peptide may be used as a lighting functional cosmetic or a pharmaceutical composition for preventing or treating a disease caused by melanin hyperpigmentation.

### SEQUENCE LISTING Free Text

An electronic file is attached.

1. A method of lightening skin, comprising:
administering a cosmetic composition comprising one or more skin-lightening functional peptides selected from:
a) an RMNE1 peptide consisting of an amino acid sequence of SEQ ID NO.: 1;
b) an RMNE1 peptide deletion variant having one to thirteen consecutive or non-consecutive amino acids deleted from the amino acid sequence of SEQ ID NO.: 1; and
c) an RMNE1 peptide deletion variant consisting of four to sixteen consecutive amino acids from the amino acid sequence of SEQ ID NO.: 1 to a subject.

2. The method of claim 1, wherein the RMNE1 peptide deletion variant consists of any one amino acid sequence selected from SEQ ID NOs: 2 to 14.

3. The method of claim 1, wherein the skin-lightening functional peptide is a combination of two or more same or different skin-lightening functional peptides.

4. The method of claim 1, wherein the skin-lightening functional peptide is characterized in that one or more of a functional molecule, a functional group, a biocompatible polymer, and a fatty acid are bound to at least one of an N-terminus and a C-terminus of the peptide.

5. The method of claim 4, wherein the functional molecule is a therapeutic peptide or therapeutic protein.

6. The method of claim 4, wherein the functional molecule is a molecule exhibiting antioxidant, anti-inflammatory or wound healing functions.

7. The method of claim 4, wherein the fatty acid is palmitoylic acid.

8. A method of preventing or treating a melanin hyperpigmentation disease, comprising:
administering a pharmaceutical composition comprising one or more skin-lightening functional peptides selected from:
a) an RMNE1 peptide consisting of an amino acid sequence of SEQ ID NO.: 1;
b) an RMNE1 peptide deletion variant having one to thirteen consecutive or non-consecutive amino acids deleted from the amino acid sequence of SEQ ID NO.: 1; and
c) an RMNE1 peptide deletion variant consisting of four to sixteen consecutive amino acids from the amino acid sequence of SEQ ID NO.: 1 to a subject,
wherein the melanin hyperpigmentation disease comprises blemishes, freckles, senile pigmentation spots, or solar lentigines.

9. The method of claim 8, wherein the RMNE1 peptide deletion variant is a peptide consisting of any one amino acid sequence selected from SEQ ID NOs: 2 to 14.

10. The method of claim 8, wherein the skin-lightening functional peptide is a combination of two or more same or different skin-lightening functional peptides.

11. The method of claim 8, wherein the skin-lightening functional peptide is characterized in that one or more of a functional molecule, a functional group, a biocompatible polymer, and a fatty acid are bound to at least one of an N-terminus and a C-terminus of the peptide.

12. The method of claim 11, wherein the functional molecule is a therapeutic peptide or therapeutic protein.

13. The method of claim 11, wherein the functional molecule is a molecule exhibiting antioxidant, anti-inflammatory or wound healing functions.

14. The method of claim 11, wherein the fatty acid is palmitoylic acid.

15. A medical device selected from among wound dressings, fillers, or composite fillers comprising one or more skin-lightening functional peptides selected from:
a) an RMNE1 peptide consisting of an amino acid sequence of SEQ ID NO.: 1;
b) an RMNE1 peptide deletion variant having one to thirteen consecutive or non-consecutive amino acids deleted from the amino acid sequence of SEQ ID NO.: 1; and
c) an RMNE1 peptide deletion variant consisting of four to sixteen consecutive amino acids from the amino acid sequence of SEQ ID NO.: 1.


SEQUENCE LISTING (14 sequences)

SEQ ID NO: 1 - protein, 17 aa, Homo sapiens
DEQERRRRRGRTRVRSE

SEQ ID NO: 2 - protein, 13 aa, Homo sapiens
RRRRRGRTRVRSE

SEQ ID NO: 3 - protein, 13 aa, Homo sapiens
DEQERRRRRGRTR

SEQ ID NO: 4 - protein, 11 aa, Homo sapiens
ERRRRRGRTRV

SEQ ID NO: 5 - protein, 4 aa, synthetic construct
DEQE

SEQ ID NO: 6 - protein, 4 aa, synthetic construct
VRSE

SEQ ID NO: 7 - protein, 4 aa, synthetic construct
GRTR

SEQ ID NO: 8 - protein, 4 aa, synthetic construct
TRVR

SEQ ID NO: 9 - protein, 4 aa, synthetic construct
QERR

SEQ ID NO: 10 - protein, 6 aa, synthetic construct
DEQERR

SEQ ID NO: 11 - protein, 6 aa, synthetic construct
GRTRVR

SEQ ID NO: 12 - protein, 8 aa, synthetic construct
GRTRVRSE

SEQ ID NO: 13 - protein, 14 aa, synthetic construct
ERRRRRGRTRVRSE

SEQ ID NO: 14 - protein, 15 aa, synthetic construct
QERRRRRGRTRVRSE